(19) United States
(12) Patent Application Publication
(10) Pub. No.: US 2010/0114326 A1
(43) Pub. Date: May 6, 2010

(54) MODULAR IMPLANT FOR JOINT PROSTHESIS

(75) Inventors: Nathan WINSLOW, Warsaw, IN (US); Thomas M. Vanasse, Warsaw, IN (US)

Correspondence Address: BURR & BROWN, PO BOX 7068, SYRACUSE, NY, 13261-7068, US

(73) Assignee: Biomet Manufacturing Corp., Warsaw, IN (US)

(21) Appl. No.: 12/608,283

(22) Filed: Oct. 29, 2009

Related U.S. Application Data
(60) Provisional application No. 61/109,643, filed on Oct. 30, 2008.

(51) Int. Cl. A61F 2/30

(52) U.S. Cl. 623/23.42

(57) ABSTRACT

A modular prosthetic implant is provided, including a support cleat unit having a circular seat portion and a shape delimited by an imaginary dome. The support cleat unit includes a cylindrical body portion having a central axis and a hole concentric with the central axis, the hole having a least a first section whose diameter defines a female Morse taper adapted to receive a male member having a corresponding Morse taper. The support cleat unit also includes a plurality of arched appendages integral with the cylindrical body portion. The arched appendages are radially spaced apart from one another and extend outwardly from a base portion thereof at an outer circumferential surface of the cylindrical body portion so that lower/outer surfaces of the arched appendages define the overall domed shape of the support cleat unit.

### FIELD OF THE INVENTION

The present invention relates to the field of orthopaedic implants, and in particular, to a modular prosthesis for use in conjunction with partial or total joint replacement procedures, such as humeral and femoral replacement and reconstruction.

### BACKGROUND OF THE INVENTION

U.S. Pat. No. 7,445,638, the entirety of which is incorporated herein, describes humeral implants that include three separate elements: anchoring stem; a support unit; and one of a cap-shaped humeral head insert or a cup shaped insert, depending on whether partial, full or reverse shoulder reconstruction is required. The stem, which is inserted in the medullary canal of the humerus, is used in conjunction with the support unit to ensure stabilization of the support unit, which, when properly stabilized, functions to anchor the humeral head implant in the humerus.

There are, however, some drawbacks associated with the prior art, and it would be desirable to overcome these drawbacks in order to improve the process and results associated with joint prostheses, particularly those for shoulders and hips.

One drawback associated with the prior art is that after the support unit is impacted in the resected humerus, the upper end of the neck that mates with the humeral head (cap or cup) insert protrudes from the surface that is otherwise flush with respect to the resected humerus. The protruding shaft part occludes the surgeon's access to the glenoid cavity, and its presence can impede the surgeon's progress with respect to other portions of the implantation process in the glenoid region before the humeral insert (cap or cup) is finally impacted thereon. It would be desirable to provide as much clearance as possible for the surgeons during the implantation process while maintaining the ability to facilitate a secure and reliable connection between the support unit and the humeral insert.

It would also be desirable to reduce the number of modular components needed for the prosthetic unit, and it would be particularly desirable to eliminate the need for the stem part traditionally implanted, for example in the medullary canal of the humerus (in shoulder applications) or the femoral canal (in hip applications).

It is an object of the present invention to improve implantation technology and the implantation process, and to overcome the drawbacks associated with prior art joint prostheses.

### SUMMARY OF THE INVENTION

The present invention achieves its objectives by providing cooperating modular prosthetic components that do not occlude the surgeon's access to other adjacent areas that require reconstruction and by reducing the number of modular components required overall. According to the present invention, this is accomplished without sacrificing the stability of the implant by virtue of the modified structure of the support unit, whose structure promotes bony ingrowth into porous portions of the support unit for secure fixation of the prosthetic joint in the humerus that can be used with, or preferably without a traditional stem part.

The present invention provides, in particular, a support cleat unit for use in conjunction with modular prosthesis units for partial, reverse or total joint reconstruction.

According to one embodiment of the present invention, a modular prosthetic implant is provided, comprising a support cleat unit having a seat portion, which is preferably circular, and having an overall shape delimited by an imaginary domed surface, which is preferably substantially hemispherical in shape with a circular base shape, but which also includes dome shapes having an oblong or oval base shape or the like, as needed for use in connection with the physiological requirements of a particular type of joint. The support cleat unit comprises a centrally located cylindrical body portion extending from a first end toward an opposed second end thereof, and which has a central axis and a hole concentric with the central axis. The hole extends from a first opening in the first end of the cylindrical body portion toward the second end of the cylindrical body portion, and has a least a first section whose diameter defines a female Morse taper adapted to receive a male member having a corresponding Morse taper.

The support cleat unit also comprises a plurality of arched appendages integral with the cylindrical body portion. The arched appendages are radially spaced apart from one another and extend radially outwardly from an outer circumferential surface of the cylindrical body portion so that outer surfaces of the arched appendages delimit a skeleton of the imaginary overall dome shape of the support cleat unit. The support cleat unit according to the present invention can be made of any suitable bio-compatible materials, examples of which include, but are not limited to titanium, cobalt, stainless steel and polyether ether ketone (PEEK).

According to one aspect of the present invention, a thickness (or width) of each arched appendage increases from the base portions thereof toward terminal ends thereof, so as to define an outwardly increasing tapered thickness that is greater at the terminal ends of said arched appendages than at the base portions thereof [see, e.g., FIGS. 9A-9D]. According to yet another aspect, the thickness/width of each arched appendage decreases from the base portions thereof toward terminal ends thereof, so as to define an outwardly decreasing tapered thickness that is less at the terminal ends of said arched appendages than at the base portions thereof [see, e.g., FIGS. 10A-10D].

According to one aspect of the present invention, the hole in the cylindrical body portion comprises a through hole extending from the first opening in the first end thereof toward an opposed second opening in the second end (seat) thereof. The first section of the hole has a terminal end spaced a distance apart from the second end of the cylindrical body, and the terminal end of the first section has an opening with a diameter that is smaller than a diameter of the first opening so as to define an inner seat. The opening of the terminal end of the first section communicates with a second section of the through hole extending from the opening of the terminal end of the first section toward the second opening at the second end of the cylindrical body portion. Preferably, at least a portion of the second section of the through hole is threaded.

According to another aspect of the present invention, the support cleat unit further comprises a cap member adapted to threadedly engage at least a portion of the second section of the through hole proximate the second end of the cylindrical body portion so as to cover the opening at the second end of the cylindrical body portion and essentially define the seat of the cylindrical body portion.

According to another embodiment of the present invention, a plurality of porous metal portions defining fin-like extensions are provided between the outer circumferential surface of the cylindrical body portion and an upper/inner surface of each arched appendage [see, e.g., FIG. 2A]. The porous metal should be a bio-compatible metal, suitable examples of which include, but are not limited to porous titanium, titanium alloy and tantalum.

According to another embodiment of the present invention, the support cleat unit further comprises a porous metal coating provided on at least a portion of the outer circumferential surface of the cylindrical body portion. Preferably, the porous metal coating is also provided on surfaces of the arched appendages. The porous metal coating can be any suitable bio-compatible porous metal coating, such as a cobalt or titanium coating, and can be applied in any known manner, such as plasma spraying.

According to yet another aspect, the porous metal coating covers substantially all of the outer surface of the cylindrical body portion and covers substantially all surfaces of the arched appendages, with the exception of a portion of the outer/lower surfaces thereof [see, e.g., FIGS. 2B-2C].

According to another embodiment of the present invention, terminal ends of each of the arched appendages further comprise a foot member extending radially outwardly with respect to the central axis of the cylindrical body portion [see, e.g., FIG. 3]. According to one aspect, an upper surface of each foot member is flush with a surface of the first end of the cylindrical body portion.

This integral structure can be accomplished by machining the appendages from a bowl-shaped support cleat unit pre-form, or via the initial mold design. These processes would be readily understood by one skilled in the art.

According to yet another embodiment of the present invention, the terminal ends of each of the arched appendages are connected to one another via a rim circumscribing or otherwise surrounding the cylindrical body portion [see, e.g., FIG. 4]. According to one aspect, an upper surface of the rim is flush with the surface of the first end of the cylindrical body portion.

According to another embodiment of the present invention, the modular implant further comprises an insert unit adapted to cooperate with a joint cavity of a patient. For example, in the case of a humeral implant, the insert unit would cooperate with the native glenoid cavity of a patient or a glenoid implant. However, it should be understood that the present invention is not strictly limited to humeral implants. The insert unit comprises a male member having a Morse taper corresponding to the female Morse taper of the first section of the hole in the cylindrical body portion of the support unit.

According to one aspect of the present invention, the thickness of each arched appendage increases from the base portions thereof toward terminal ends thereof, so as to define an outwardly increasing tapered thickness that is greater at the terminal ends of the arched appendages than at the base portions thereof.

According to another aspect of the present invention, the thickness of each the arched appendage decreases from the base portions thereof toward terminal ends thereof, so as to define an outwardly decreasing tapered thickness that is less at the terminal ends of the arched appendages than at the base portions thereof.

According to one aspect of the present invention, a porous metal coating is provided on at least a portion of the outer circumferential surface of the cylindrical body portion and substantially all surfaces of the arched appendages. According to another aspect, the porous metal coating covers substantially all of the outer surface of the cylindrical body portion and covers substantially all surfaces of the arched appendages. According to yet another aspect, the porous metal coating on the arched appendages includes a plurality of barbed structures formed thereon

### BRIEF DESCRIPTION OF THE DRAWINGS

FIG. 1A is a perspective view of a support cleat unit according to one embodiment of the present invention, FIG. 1B is a top plan view of the support unit shown in FIG. 1A, FIG. 1C is a bottom plan view of the support unit shown in FIG. 1A, and FIG. 1D is a cross-sectional view taken through line A-A in FIG. 1A. FIG. 1E is a perspective view of another support cleat unit according to the present invention, and FIG. 1F is a cross-sectional view of the support cleat shown in FIG. 1E, showing an internal structure of the cylindrical body portion that is different from that shown and described in connection with FIGS. 1A-1D.

FIG. 2A is a perspective view of a support cleat unit including a porous metal fin structure unit according to one aspect of the present invention, and FIGS. 2B and 2C are a front view and a perspective view, respectively, of a support unit having a different porous metal coating structure according to another aspect of the present invention.

FIG. 3 is a perspective view of a support cleat unit according to another embodiment of the present invention.

FIG. 4 is a perspective view of a support cleat unit according to another embodiment of the present invention.

FIGS. 5A and 5B are views showing a modular prosthesis assembly, in the specific context of an example for shoulder prosthesis, including the support cleat unit shown in FIG. 1A and a male (cap) humeral head insert unit adapted to cooperate with the native glenoid cavity or reconstructed glenoid support.

FIGS. 5C-5E are views showing a male (cap) humeral head insert unit having a male Morse taper shaft extending from the bottom surface thereof. In FIGS. 5C and 5D, the axis of the shaft is concentric with the central axis of the cap portion. In FIG. 5E, the axis of the shaft is eccentric with respect to the central axis of the cap portion.

FIG. 6 is an exploded view showing a modular shoulder prosthesis assembly including the support cleat unit shown in FIG. 1A and a female (cup) unit adapted to cooperate with a male (cap) glenoid implant (not shown).

FIGS. 7A-7C show a support cleat unit according to another embodiment of the present invention utilized in conjunction with the hip in the case of a partial femoral head removal.

FIGS. 8A-8C show a support cleat unit according to another embodiment of the present invention utilized in conjunction with the hip in the case of a total femoral head and neck removal.

FIGS. 9A-9D are front and perspective views showing a support cleat unit including arched appendages having a tapered structure according to another aspect of the present invention, whereby the thickness of the terminal ends 31 of the arched appendages is greater than that of the base portions thereof that extend from the cylindrical body 2 proximate the second end 22 thereof.

FIGS. 10A-10D are front and perspective views showing a support cleat unit including arched appendages having a tapered structure according to another aspect of the present invention, whereby the thickness of the terminal ends 31 of the arched appendages is less than that of the base portions thereof that extend from the cylindrical body 2 proximate the second end 22 thereof.

### DETAILED DESCRIPTION OF THE INVENTION

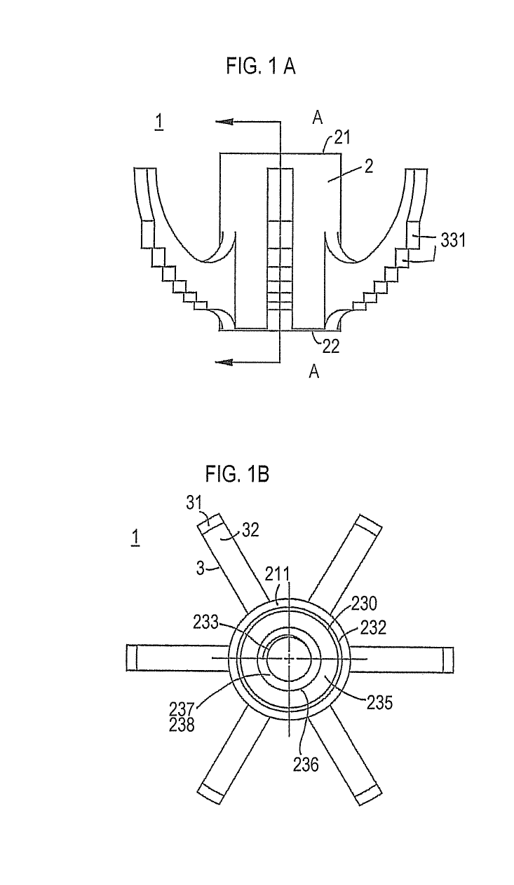

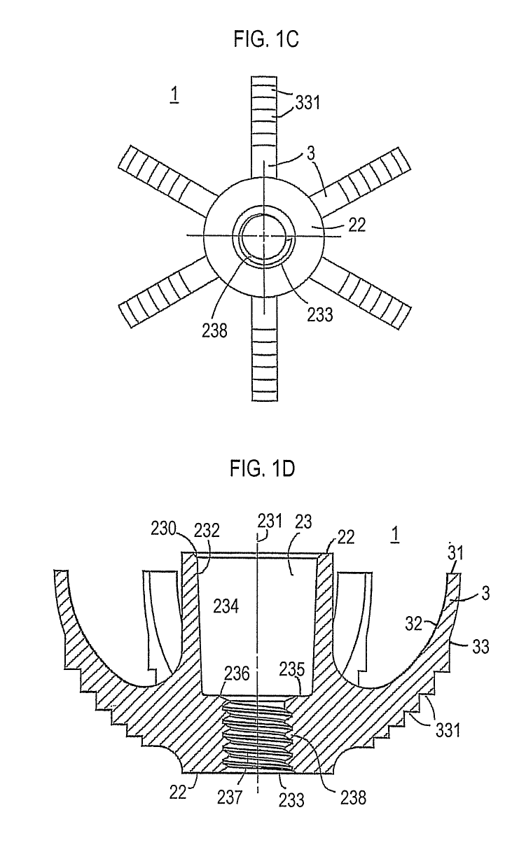

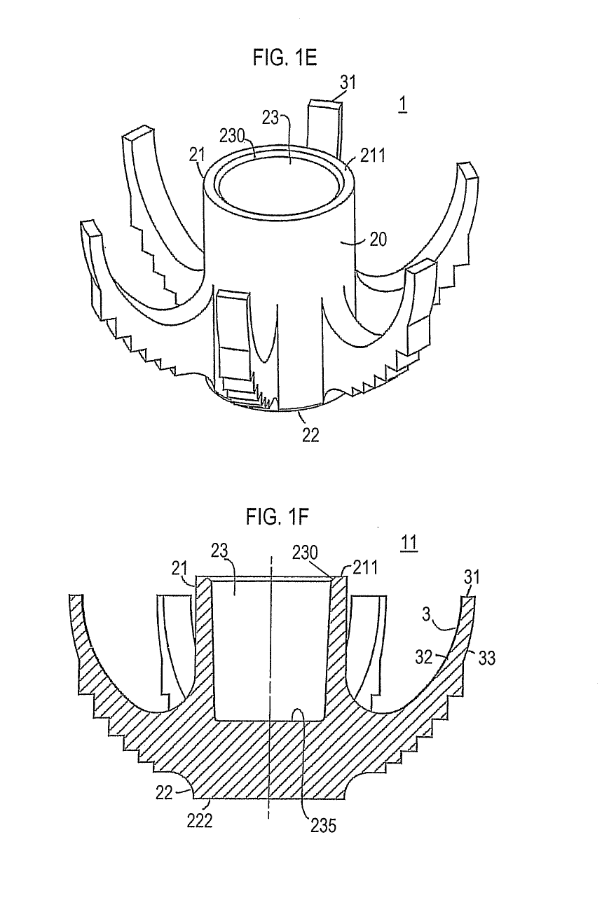

FIG. 1A is a perspective view of a support cleat unit according to one embodiment of the present invention. FIG. 1B is a top plan view of the support unit shown in FIG. 1A, FIG. 1C is a bottom plan view of the support unit shown in FIG. 1A, and FIG. 1D is a cross-sectional view taken through line A-A in FIG. 1A. FIG. 1E is a perspective view of another support cleat unit according to the present invention, and FIG. 1F is a cross-sectional view of the support cleat shown in FIG. 1E, showing an internal structure of the cylindrical body portion that is different from that shown and described in connection with FIGS. 1A-1D.

The support cleat unit 1 includes a seat portion 22, which is preferably circular, and has an overall shape delimited by an imaginary domed surface, which is preferably substantially hemispherical in shape with a circular base shape, but which also includes dome shapes having an oblong or oval base shape or the like, as dictated by the physiological requirements of a particular type of joint. The support cleat unit 1 comprises a centrally located cylindrical body portion 2 extending from a first end 21 toward an opposed second end 22 (e.g., the seat portion) thereof, and which has a central axis 231 and a hole 23 concentric with the central axis 231. As shown in FIGS. 1D and 1F, the hole 23 extends from a first opening 232 in the first end 21 of the cylindrical body portion 2 toward the second end 22 of the cylindrical body portion 2, and has a least a first section 234 whose diameter defines a female Morse taper adapted to receive a male member having a corresponding Morse taper.

According to one aspect of the present invention, as shown in FIG. 1D, the hole 23 in the cylindrical body portion 2 comprises a through hole extending from the first opening 232 in the first end 21 thereof toward an opposed second opening 233 in the second end (seat) 22 thereof. The first section 234 of the hole 23 has a terminal end 235 spaced a distance apart from the second end 22 of the cylindrical body 2, and the terminal end 235 of the first section 234 has an opening 236 with a diameter that is smaller than a diameter of the first opening 232 so as to define an inner seat (at terminal end 235). The opening 236 of the terminal end 235 of the first section 23 communicates with a second section 237 of the through hole 23 extending from the opening 236 of the terminal end 235 of the first section 234 toward the second opening 233 at the second end 22 of the cylindrical body portion 2. Preferably, at least a portion of the second section 237 of the through hole 23 is threaded.

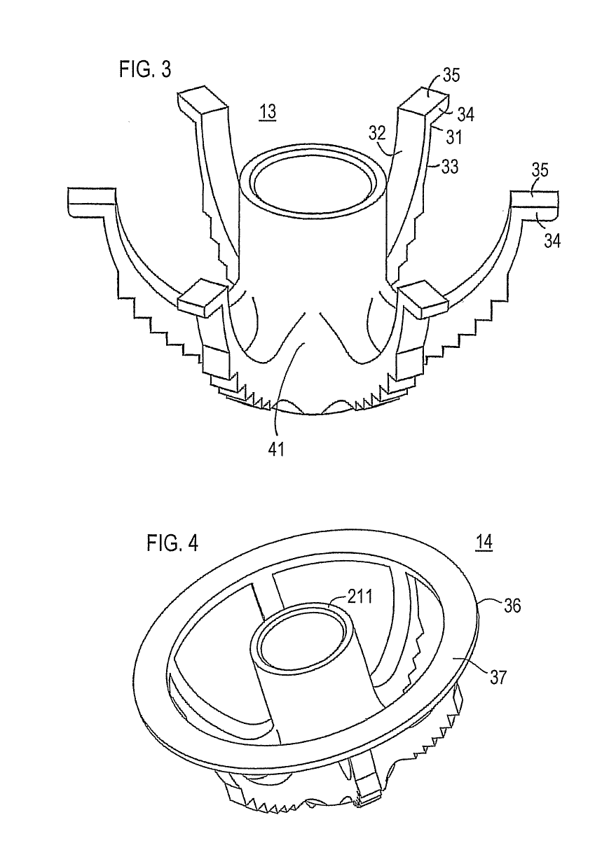

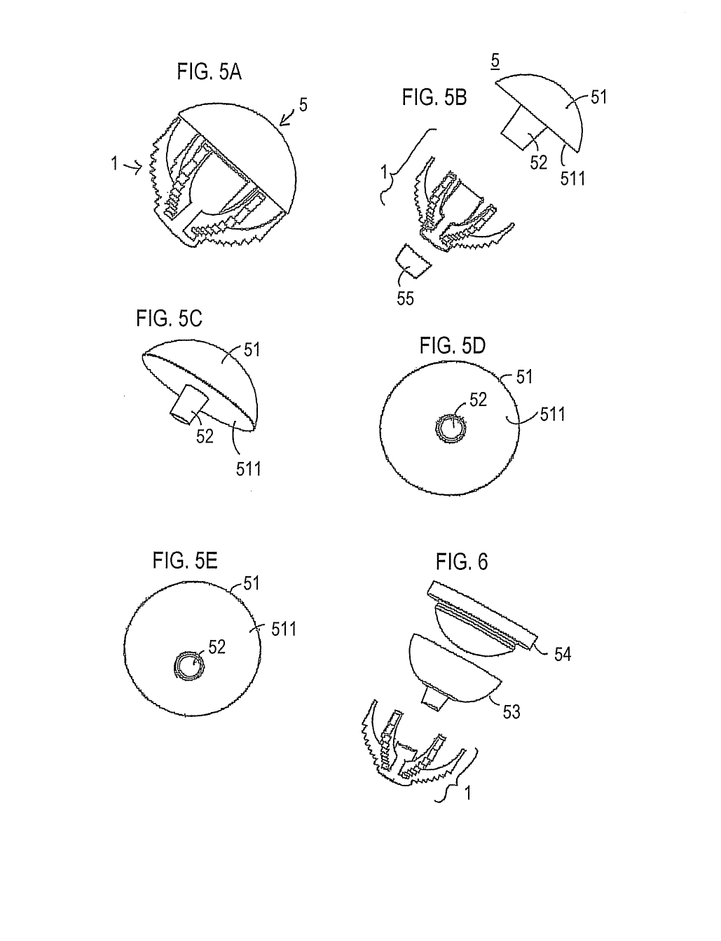

For example, in FIGS. 1B-1D, a threaded section 238 of the through hole 23, having a smaller diameter than that of the first section 234 defining the reverse Morse taper, is provided at the second end of the cylindrical body portion 2, which extends through the second end 22 thereof and has a dual purpose. One purpose of this feature is to facilitate the insertion of instrumentation for insertion during implantation and extraction, if necessary, of the support cleat unit. Another purpose is to facilitate means for affixing an end cap 55 (see, e.g., FIG. 5B) onto the second end 22 of device. This type of end cap 55, which may be made of a porous metal, such as REGENEREX®, or a porous coated metal, serves to prevent fluid and bone from entering into the device in situ and serves to anchor the device into the bone. Alternatively, traditional stem could be attached to the support unit at this location, as well.

The support cleat unit 1 also comprises a plurality of arched appendages 3 that are integral with respect to the cylindrical body portion 2. The arched appendages 3 are radially spaced apart from one another and extend radially outwardly from an outer circumferential surface 20 of the cylindrical body portion 2 so that outer/lower surfaces 33 of the arched appendages delimit a skeleton of the imaginary overall dome shape of the support cleat unit 1.

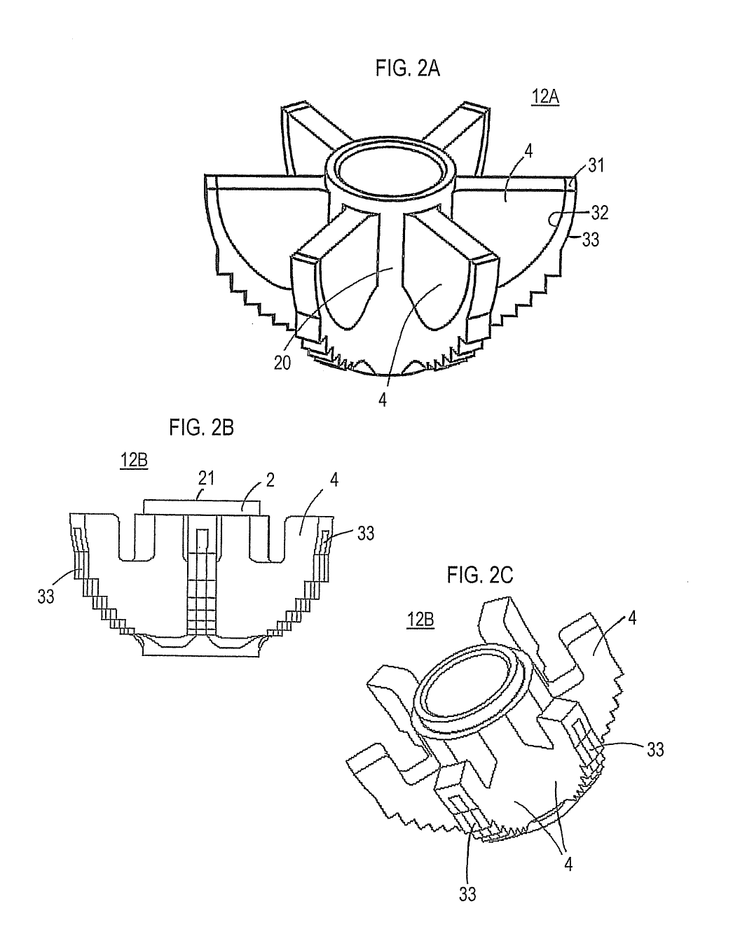

FIG. 2A is a perspective view of a support cleat unit 12A including a porous metal fin structure unit according to one aspect of the present invention. As shown in FIG. 2A, a plurality of porous metal portions 4 defining fin-like extensions are provided and extend between the outer circumferential surface 20 of the cylindrical body portion 2 and an upper/inner surface 32 of each arched appendage 3. The porous metal material must be a bio-compatible metal, suitable examples of which include, but are not limited to Regenerex®, porous titanium, titanium alloy and tantalum. Although it is not necessary to provide the porous metal extensions, the provision of the fin-like extension structure serves to help securely anchor the device into the bone and promote bone re-growth.

FIGS. 2B and 2C are a front view and a perspective view, respectively, of a support cleat unit having a different porous metal coating structure according to another aspect of the present invention. As shown, the substantially the entire outer surface of the support cleat unit 12B is coated with a thick porous metal material coating 4, with the exception of portions of the outer surface of the first 21 and second 22 ends of the cylindrical body portion 2 and the outer/lower surfaces 33 of the arched appendages 3. After the porous metal material 4 is provided on the support cleat unit 12B as a substrate, the porous metal material is machined to have a corresponding shape and to define fin-like extensions between the arched appendages 3 and the cylindrical body portion 2, covering the central portion of the cylindrical body portion 2 and nearly the entire surface of the arched appendages 3, with the exceptions described above. Again, the provision of the porous metal material coated structure serves to help securely anchor the device into the bone and promote bony ingrowth.

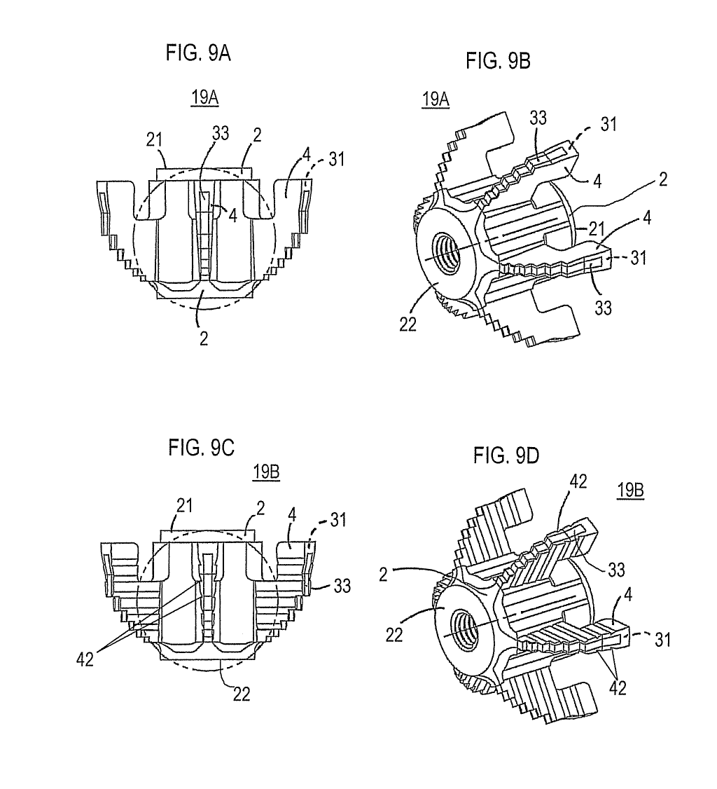

FIGS. 9A-9D are front and perspective views showing support cleat units 19A and 19B including arched appendages having a tapered structure according to additional aspects of the present invention, whereby the thickness or width of the arched appendages decreases toward the base so that the thickness of the covered terminal ends 31 of the arched appendages is greater than that of the base portions thereof that extend from the cylindrical body 2 proximate the second end 22 thereof. The advantages associated with this aspect of the present invention are as follows.

The outer diameter of the support cleat unit 19A, as defined by the outermost surfaces of the arched appendages 3, is smaller proximate the second end 22 of the cylindrical body portion 2 (the end of the device which is initially inserted into the prepared bone). Accordingly, the diameter of the portion of bone that is prepared to receive the implant can be smaller as well, and as the support cleat unit 19A is impacted into the prepared bone, the increasing diameter provides additional compressive forces on the surrounding bone structure, creating a wedge-fit in the bone. This increased compressive force triggers a Wolfe's law response, whereby the bone reacts to the force by strengthening in that area, thereby improving the strength and bone regrowth in the implant area.

As shown, the outer surface of the support cleat units 19A, 19B are substantially entirely coated with a porous metal material coating 4, as described above in connection with FIGS. 2B and 2C, with the exception of portions of the first 21 and second 22 ends of the cylindrical body portion 2 and portions of the outer surfaces 33 of the arched appendages 3, as described above. The provision of the porous metal coating 4 promotes bony ingrowth and aids in securely affixing the support cleat unit 19A, 19B in the resected portion of the respective bone (e.g., humerus or femur). The porous metal coating 4 on the arched appendages is machined to have a tapered structure corresponding to that of the underlying support unit substrate.

The main difference between the support cleat unit 19A in FIGS. 9A and 9B and the support cleat unit 19B in FIGS. 9C and 9D is that a plurality of notches defining a barbed structure 42 are machined into the outer surface of the porous metal material coated arched appendages in support cleat unit 19B. The barbed structures 42 facilitate improved interdigitation between the resected bone and the arched appendages to further improve bone regrowth in conjunction with the implant.

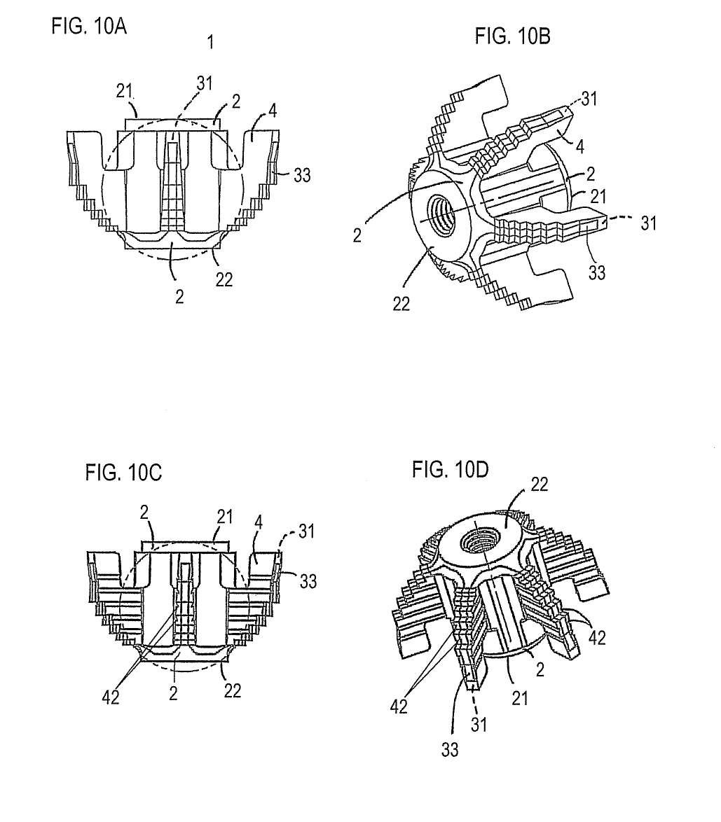

FIGS. 10A-10D are front and perspective views showing support cleat units 10A and 10B including arched appendages having a tapered structure according to another aspect of the present invention, whereby the thickness of the arched appendages increases toward the bases thereof, so that the thickness of the terminal ends 31 of the arched appendages is less than that of the base portions thereof that extend from the cylindrical body 2 proximate the second end 22 thereof. When this embodiment is used, the bone is prepared to accept insertion of the wider diameter portion of the support cleat unit 10A, 10B, and bone graft is packed into the slots in the portions of the prepared bone where the arched appendages become thinner to compensate for the smaller outer diameter of the support cleat unit 10A, 10B. Once bone is grown around the implant, the wider base of the appendages improves the stability and increases the pull-out strength.

As shown, the outer surfaces of the support cleat units 10A, 10B are also coated with the porous metal material coating 4, as described above in connection with FIGS. 2B and 2C and FIGS. 9A-9D. In addition, the barbed structure 42 is provided in conjunction with the outer surfaces of the coated arched appendages of the support cleat unit 10B shown in FIGS. 10C and 10D.

According to another embodiment, the support cleat unit includes a thinner porous metal coating 41 provided on at least a portion of the outer circumferential surface 20 of the cylindrical body portion 2 (see, e.g., FIG. 3). The provision of the porous metal coating 41 promotes bony ingrowth and aids in securely affixing the support cleat unit 1 in the resected portion of the respective bone (e.g., humerus or femur). This porous metal coating is provided as an alternative to the embodiment shown in FIG. 2A, because plasma spraying an additional porous metal coating layer onto the REGENEREX® can block the pore structure and be counter effective.

Preferably, the porous metal coating 41 is also provided on surfaces of the arched appendages 3. The porous metal coating 41 can be any suitable bio-compatible porous metal coating, such as REGENEREX®, a cobalt or a titanium coating, and can be applied in any known manner, such as plasma spraying. It is also possible to use a hydroxyapatite (HA) coating over the outer surfaces of the cylindrical body portion 2 and the arched appendages 3.

FIG. 3 is a perspective view of a support cleat unit 13 according to another embodiment of the present invention. As shown, the terminal ends 31 of each of the arched appendages 3 include a foot member 34 extending radially outwardly with respect to the central axis of the cylindrical body portion. The upper surface 35 of each foot member 34 is flush or slightly lower than the surface 211 of the first end 21 of the cylindrical body portion 2.

The provision of the foot members 34 serves as an anti-subsidence mechanism and contributes to ensuring that the support cleat unit 13 is securely implanted in the resected portion of the humerus or femur, for example. Since the foot members 34 are slightly lower than or substantially flush with respect to the upper surface 211 of the first end 21 of the cylindrical body portion 2, the benefits described above remain attainable, and the foot members 34 do not obscure the implant site or hinder the surgeon's access to the necessary locations during the surgical implantation.

FIG. 4 is a perspective view of a support cleat unit 14 according to another embodiment of the present invention. As shown, the terminal ends 31 of each of the arched appendages 3 are circumferentially connected to one another via a rim 36 circumscribing or otherwise substantially surrounding the cylindrical body portion 2. The upper surface 37 of the rim 36 is slightly lower than or substantially flush with the surface 211 of the first end 21 of the cylindrical body portion 2. Like the embodiment shown in FIG. 3, the rim 36 structure provides an anti-subsidence mechanism, and the position of the rim surface 37 with respect to the upper surface 211 of the cylindrical body portion 2 (e.g., slightly lower than or flush) provides the benefits described above in a similar fashion.

FIGS. 5A and 5B are views showing a modular prosthetic assembly including the support cleat unit shown in FIG. 1A and a male (cap) humeral head unit adapted to cooperate with the joint cavity, such as the glenoid cavity, or a reconstructed joint cavity support, such as a reconstructed glenoid support. One skilled in the art should readily appreciate that this structure could easily translate to relate to a hip implant. FIGS. 5C-5E are views showing a male insert unit 5 defining a humeral head unit having a cap portion 51 and a male Morse taper shaft 52 extending from the bottom surface 511 thereof.

In FIGS. 5C and 5D, the axis of the Morse taper shaft 51 is concentric with a central axis of the cap portion 51 of the male head insert 5. In FIG. 5E, the axis of the shaft 51 is eccentric with respect to the central axis of the cap portion 51. An adjustable humeral head having a male Morse taper shaft, such as that described in U.S. Pat. No. 6,492,699, the entirety of which is incorporated herein by reference, can also be used in conjunction with the support cleat units according to the present invention.

The head assembly disclosed in the '699 patent includes a head and an adaptor. The adaptor is rotated to achieve a certain amount of offset between the head and the axis of the Morse taper. The ability to vary the degree of offset is desired so as to achieve complete coverage of the resected humerus, even in situations where the support unit is not centered in the resected surface of the humerus.

An end cap member 55 is also provided, which is adapted to threadedly engage at least a portion of the second section 237 (e.g., the threaded portion 238) of the through hole 23 proximate the second end 22 of the cylindrical body portion 2 so as to cover the opening 233 at the second end of the cylindrical body portion 2 and, in that case, essentially define the seat of the cylindrical body portion 2.

FIG. 6 is an exploded view showing a modular prosthetic assembly including the support cleat unit 1 shown in FIG. 1A and a female (cup) unit 53 adapted to cooperate with a (cup) 54. As explained above, with respect to a shoulder application, the support cleat unit 1 is impacted in the resected humerus, and an insert unit 51 is attached thereto which has a shape to replicate the humeral head (male) that articulates with the glenoid cavity, or a female cup-shaped cap 53 that articulates with a male cup implanted in the glenoid cavity. As mentioned above, however, the present invention is not strictly limited to shoulder joint applications, and can be used in conjunction with hip Arthroplasty, as explained in more detail below, or with other Arthroplasty applications, as needed.

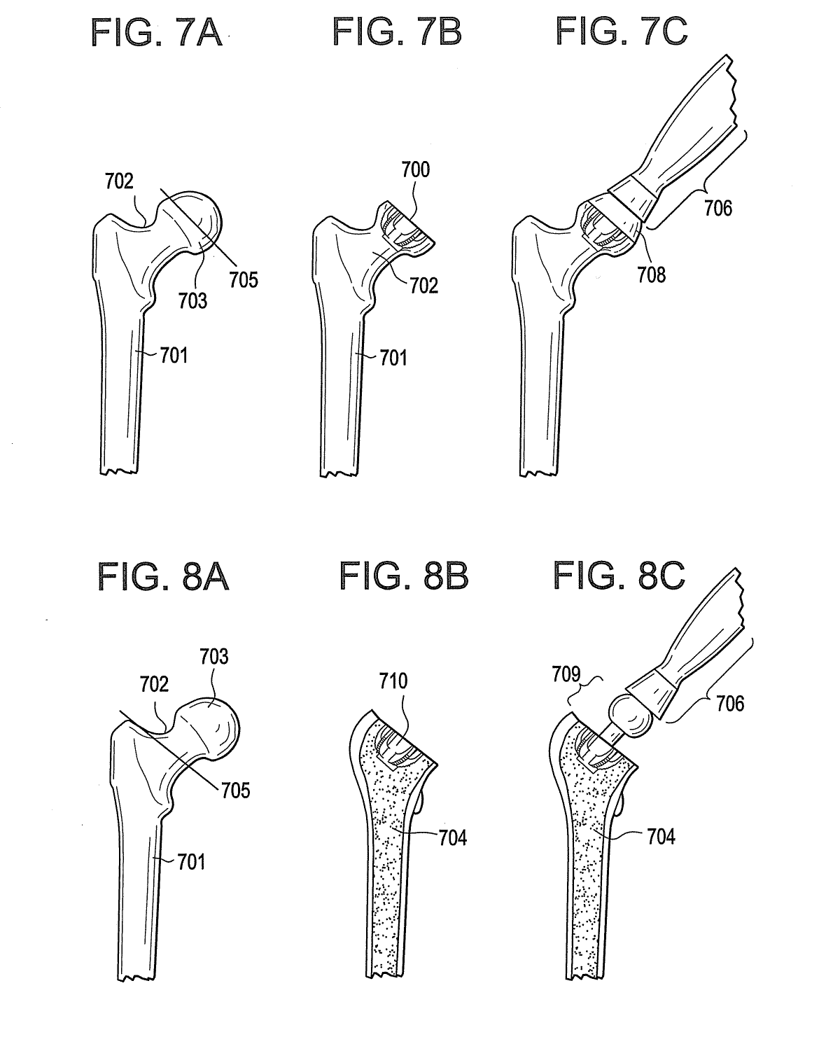

FIGS. 7A-7C and 8A-8C illustrate methods for utilizing the support cleat unit 1 in the hip. In particular, FIGS. 7A-7C show a partial femoral head removal situation and FIGS. 8A-8C show a complete femoral head and neck removal. In both cases, these components could also be used in conjunction with an acetabular implant in total hip Arthroplasty (THA).

As shown in FIG. 7A, a portion of the femoral head 703 is resected in a location shown, for example, by the line 705 representing the resection level, and after the required preparation through broaching or reaming, an appropriately sized support cleat unit is inserted into the femoral head 703 (see FIG. 7B). A hemispherical head implant unit 708 is then impacted onto the cleat, and which articulates with the acetabulum, using an impacting tool 706, as shown in FIG. 7C. Like the case with the shoulder, the head implant is made from an appropriate biocompatible material, such as a ceramic, metal, polyether ether ketone (PEEK), or the like.

With respect to the complete head and neck removal situation illustrated in FIGS. 8A-C, the femoral head 703 and neck 702 are resected in FIG. 8A in a manner that is consistent with the procedure required in connection with current stemmed hip implants, and after proper preparation, the support cleat unit 1 is impacted into femur, as shown in FIG. 8B. A femoral neck and head implant 709, which can be a single implant component or a combination of separate implant components, and which articulates with the acetabulum, is then impacted onto the support cleat unit 1 to complete the replacement (see FIG. 8C).

According to the present state of the art, in order to perform a THA with current resurfacing devices, a large incision is required to allow access to the acetabulum. With all or part of the femoral head removed, which is made possible in connection with the support cleat according to the present invention, it is possible to ream the acetabulum through a smaller incision since the femoral head in not blocking the surgeon's view and access. This significant benefit can be readily appreciated as an important and valuable advancement with respect to both the surgical and healing processes.

As one skilled in the art can appreciate, the overall shape of the support cleat unit used in conjunction with hip applications should be slightly different than that described above in connection with shoulder applications, based on physiological constraints, and in order to properly fit the support basket into the resected femoral neck/femur and provide adequate stability. Suitable modifications may be made with respect to the number of arched appendages provided (e.g., 4 or 8) or with respect to the overall imaginary dome shape delimited by the arched appendages (e.g., an oval or oblong base, rather than circular, in hip applications).

Based on the foregoing descriptions, one skilled in the art should readily understand how to make, use and/or modify the support cleat unit described as needed for use in hip Arthroplasty. As noted, any such modifications would indeed be minimal, and no additional drawings are required to illustrate the number of different possibilities that are within the scope of the present invention.

What is claimed is:

1. A modular prosthetic implant comprising:
a support cleat unit having a circular seat portion and a shape delimited by an imaginary dome, said support cleat unit comprising:
a cylindrical body portion extending from a first end toward an opposed second end thereof defining said seat portion of said support cleat unit, said cylindrical body portion having a central axis and a hole concentric with said central axis, said hole extending from a first opening in said first end of said cylindrical body portion toward said second end of said cylindrical body portion, said hole having a least a first section whose diameter defines a female Morse taper adapted to receive a male member having a corresponding Morse taper; and
a plurality of arched appendages integral with said cylindrical body portion, said arched appendages being radially spaced apart from one another and extending outwardly from a base portion thereof at an outer circumferential surface of said cylindrical body portion so that lower/outer surfaces of said arched appendages define the overall domed shape of said support cleat unit.

2. The modular prosthetic implant according to claim 1, further comprising a plurality of discrete porous metal portions spanning between said outer circumferential surface of said cylindrical body portion and an upper/inner surface of said arched appendages.

3. The modular prosthetic implant according to claim 1, wherein terminal ends of each of said arched appendages further comprise a foot member extending radially outwardly with respect to said central axis of said cylindrical body portion.

4. The modular prosthetic implant according to claim 3, wherein an upper surface of each said foot member is flush with a surface at said first end of said cylindrical body portion.

5. The modular prosthetic implant according to claim 1, wherein terminal ends of each said arched appendage are circumferentially connected to one another via a rim circumscribing said cylindrical body portion.

6. The modular prosthetic implant according to claim 5, wherein an upper surface of said rim is flush with a surface at said first end of said cylindrical body portion.

7. The modular prosthetic implant according to claim 1, further comprising an insert unit adapted to cooperate with one of a receptacle portion of a joint cavity of a patient and a joint cavity implant provided in a receptacle portion of a joint cavity of a patient, said insert unit at least comprising a male member having a Morse taper corresponding to said female Morse taper of said first section of said hole in said cylindrical body portion of said support cleat unit.

8. The modular prosthetic implant according to claim 1, further comprising a porous metal coating provided on at least a portion of said outer circumferential surface of said cylindrical body portion.

9. The modular prosthetic implant according to claim 8, wherein said porous metal coating is further provided on surfaces of said arched appendages.

10. The modular prosthetic implant according to claim 1, wherein said hole of said cylindrical body portion comprises a through hole extending through said cylindrical body portion from said first opening in said first end thereof toward an opposed second opening in said second end thereof said.

11. The modular prosthetic implant according to claim 10, wherein said first section of said hole has a terminal end spaced a distance from said second end of said cylindrical body, wherein said terminal end of said first section has an opening diameter that is smaller than a diameter of said first opening so as to define a seat, and wherein said opening of said terminal end of said first section communicates with a second section of said through hole extending from said opening of said terminal end of said first section toward a second opening at said second end of said cylindrical body portion.

12. The modular prosthetic implant according to claim 11, wherein said second section of said through hole is threaded.

13. The modular prosthetic implant according to claim 12, further comprising a plug member threadedly engaging at least a portion of said second section of said through hole proximate said second end of said cylindrical body portion.

14. The modular prosthetic implant according to claim 1, wherein a thickness of each said arched appendage increases from said base portions thereof toward terminal ends thereof, so as to define an outwardly increasing tapered thickness that is greater at said terminal ends of said arched appendages than at said base portions thereof.

15. The modular prosthetic implant according to claim 1, wherein a thickness of each said arched appendage decreases from said base portions thereof toward terminal ends thereof, so as to define an outwardly decreasing tapered thickness that is less at said terminal ends of said arched appendages than at said base portions thereof

16. The modular prosthetic implant according to claim 14, further comprising a porous metal coating provided on at least a portion of said outer circumferential surface of said cylindrical body portion and substantially all surfaces of said arched appendages.

17. The modular prosthetic implant according to claim 15, further comprising a porous metal coating provided on at least a portion of said outer circumferential surface of said cylindrical body portion and on substantially all surfaces of said arched appendages.

18. The modular prosthetic implant according to claim 16, wherein said porous metal coating on said arched appendages includes a plurality of barbed structures formed thereon.

19. The modular prosthetic implant according to claim 17, wherein said porous metal coating on said arched appendages includes a plurality of barbed structures formed thereon.

20. The modular prosthetic implant according to claim 17, wherein said porous metal coating covers substantially all of the outer surface of said cylindrical body portion.
